(19) United States
(12) Patent Application Publication
(10) Pub. No.: US 2007/0293493 A1
(43) Pub. Date: Dec. 20, 2007

(54) OXAZOLIDINONE ANTIBIOTICS AND DERIVATIVES THEREOF

(76) Inventor: Milton Hammond, Branchburg, NJ (US)

Correspondence Address: MCDERMOTT WILL & EMERY LLP, 600 13TH STREET, N.W., WASHINGTON, DC, 20005-3096, US

(21) Appl. No.: 10/559,868

(22) PCT Filed: Jun. 29, 2004
(86) PCT No.: PCT/US04/20738

§ 371 Date: Mar. 2, 2007

Related U.S. Application Data
(60) Provisional application No. 60/483,901, filed on Jul. 2, 2003.
(60) Provisional application No. 60/546,985, filed on Feb. 24, 2004.

(51) Int. Cl. A61K 31/5377; A61K 31/4439; A61K 31/422; C07D 413/14

(52) U.S. Cl. 514/235.5; 514/340; 514/376; 548/229; 544/137; 546/271.4

(57) ABSTRACT

This invention relates to new oxazolidinones having a cyclopropyl moiety, which are effective against aerobic and anerobic pathogens such as multi-resistant staphylococci, streptococci and enterococci, Bacteroides spp., Clostridia spp. species, as well as acid-fast organisms such as Mycobacterium tuberculosis and other mycobacterial species.
The compounds are represented by structural formula I: 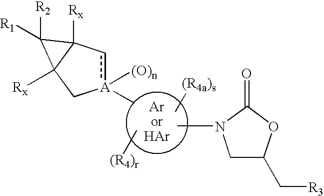 its enantiomer, diastereomer, or pharmaceutically acceptable salt or ester thereof.

### CROSS REFERENCE TO RELATED APPLICATIONS

This application claims the benefit of U.S. Provisional Application No. 60/483,901, filed Jul. 2, 2003, entitled OXAZOLIDINONE ANTIBIOTICS AND DERIVATIVES THEREOF and U.S. Provisional Application 60/546,985, filed Feb. 24, 2004, entitled OXAZOLIDINONE ANTIBIOTICS AND DERIVATIVES THEREOF, which are hereby incorporated herein by reference in their entirety.

### BACKGROUND OF THE INVENTION

Oxazolidinones represent the first new class of antibacterials to be developed since the quinolones. The oxazolidinones are synthetic antibacterial compounds that are orally or intravenously active against problematic multidrug resistant Gram positive organisms and are not cross-resistant with other antibiotics. See Riedl et al, Recent Developments with Oxazolidinone Antibiotics, Exp. Opin. Ther. Patents (1999) 9(5), Ford et al., Oxazolidinones: New Antibacterial Agents, Trends in Microbiology 196 Vol. 5, No. 5, May 1997 and WO 96/35691. See also WO 03/063862, WO 01/81350, WO 01/94342, WO 03/072553, EP 0352781 and U.S. Pat. Nos. 5,565,571 and 470537593.

This invention relates to new oxazolidinones having a cyclopropyl moiety, which are effective against aerobic and anerobic pathogens such as multi-resistant staphylococci, streptococci and enterococci, Bacteroides spp., Clostridia spp. species, as well as acid-fast organisms such as Mycobacterium tuberculosis and other mycobacterial species.

### SUMMARY OF THE INVENTION

The present invention relates to compounds of formula I: 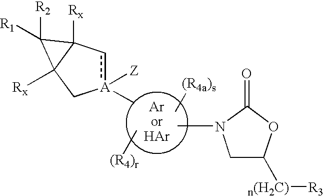
its enantiomer, diastereomer, or pharmaceutically acceptable salt, hydrate or prodrug thereof wherein:
R₁ and R₂ independently represent
hydrogen, NR₅R₆, CR₇R₈R₉, C(R)₂OR₁₄, CH₂NHR₁₄, C(═O)R₁₃, C(═NOH)H, C(═NOR₁₃)H, C(═NOR₁₃)R₁₃, C(═NOH)R₁₃, C(═O)N(R₁₃)₂, C(═NOH)N(R₁₃)₂, NHC(═X₁)N(R₁₃)₂, (C═NH)R₇, N(R₁₃)C(═X₁)N(R₁₃)₂, COOR₁₃, SO₂R₁₄, N(R₁₃)SO₂R₁₄, N(R₁₃)COR₁₄, (C₁₋₆alkyl)CN, CN, CH═C(R)₂, C(R₄)₂X₁SiR₁₆, (CH₂)ₚOH, C(═O)CHR₁₃, C(═NR₁₃)R₁₃, NR₁₀C(═X₁)R₁₃; or C₅₋₁₀ heterocycle optionally substituted with 1-3 groups of R₇, which may be attached through either a carbon or a heteroatom;
Z represents (O)ₙ, H, OH, or halogen;
A represents C (when [symbol] is present provided Z=(O)ₙ and n=0), C (when [symbol] is not present provided Z is H, OH or halogen), or N (when [symbol] is not present and Z=(O)ₙ and n=1); [symbol]
represents a bond; 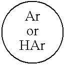
represents aryl or heteroaryl, heterocycle, heterocyclyl or heterocyclic, provided that in the case of a heteroaryl, heterocycle, heterocyclyl or heterocyclic, a cyclopropyl is not attached to a nitrogen atom on the ring;
Rₓ represents hydrogen or C₁₋₆ alkyl;
R₃ represents 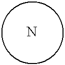
which is an optionally substituted aromatic heterocyclic group containing at least one nitrogen in the ring and which is attached through a bond on any N, and which is unsubstituted or contains 1 to 3 substituents of R₇
R₄ and R₄ₐ independently represent
hydrogen,
halogen,
C₁₋₆ alkoxy, or
C₁₋₆ alkyl
r and s independently are 1-3, with the provision that when (R₄ₐ)ₛ and (R₄)ᵣ are attached to an Ar or HAr ring the sum of r and s is less than or equal to 4.
R₅ and R₆ independently represent
hydrogen,
C₁₋₆ alkyl optionally substituted with 1-3 groups of halogen, CN, OH, C₁₋₆ alkoxy, amino, imino, hydroxyamino, alkoxyamino, C₁₋₆ acyloxy, C₁₋₆ alkylsulfenyl, C₁₋₆ alkylsulfinyl, C₁₋₆ alkylsulfonyl, aminosulfonyl, C₁₋₆ alkylaminosulfonyl, C₁₋₆ dialkylaminosulfonyl, 4-morpholinylsulfonyl, phenyl, pyridine, 5-isoxazolyl, ethylenyloxy, or ethynyl, said phenyl and pyridine optionally substituted with 1-3 halogen, CN, OH, CF₃, C₁₋₆ alkyl or C₁₋₆ alkoxy;
C₁₋₆ acyl optionally substituted with 1-3 groups of halogen, OH, SH, C₁₋₆ alkoxy, naphthaleneoxy, phenoxy, amino, C₁₋₆ acylamino, hydroxylamino, alkoxylamino, C₁₋₆ acyloxy, aralkyloxy, phenyl, pyridine, C₁₋₆ alkylcarbonyl, C₁₋₆ alkylamino, C₁₋₆ dialkylamino, C₁₋₆ hydroxyacyloxy, C₁₋₆ alkylsulfenyl, phthalimido, maleimido, succinimido, said phenoxy, phenyl and pyridine optionally substituted with 1-3 groups of halo, OH, CN, C₁₋₆ alkoxy, amino, C₁₋₆ acylamino, CF₃ or C₁₋₆ alkyl;
C₁₋₆ alkylsulfonyl optionally substituted with 1-3 groups of halogen, OH, C₁₋₆ alkoxy, amino, hydroxylamino, alkoxylamino, C₁₋₆ acyloxy, or phenyl; said phenyl optionally substituted with 1-3 groups of halo, OH, C₁₋₆ alkoxy, amino, C₁₋₆ acylamino, CF₃ or C₁₋₆ alkyl;
arylsulfonyl optionally substituted with 1-3 of halogen, C₁₋₆ alkoxy, OH or C₁₋₆ alkyl;
C₁₋₆ alkoxycarbonyl optionally substituted with 1-3 of halogen, OH, C₁₋₆ alkoxy, C₁₋₆ acyloxy, or phenyl, said phenyl optionally substituted with 1-3 groups of halo, OH, C₁₋₆ alkoxy, amino, C₁₋₆ acylamino, CF₃ or C₁₋₆ alkyl;
aminocarbonyl, C₁₋₆ alkylaminocarbonyl or C₁₋₆ dialkylaminocarbonyl, said alkyl groups optionally substituted with 1-3 groups of halogen, OH, C₁₋₆ alkoxy or phenyl five to six membered heterocycles optionally substituted with 1-3 groups of halogen, OH, CN, amino, C₁₋₆ acylamino, C₁₋₆ alkylsulfonylamino, C₁₋₆ alkoxycarbonylamino, C₁₋₆ alkoxy, C₁₋₆ acyloxy or C₁₋₆ alkyl, said alkyl optionally substituted with 1-3 groups of halogen, or C₁₋₆ alkoxy;
C₃₋₆ cycloalkylcarbonyl optionally substituted with 1-3 groups of halogen, OH, C₁₋₆ alkoxy or CN;
benzoyl optionally substituted with 1-3 groups of halogen, OH, C₁₋₆ alkoxy, C₁₋₆ alkyl, CF₃, C₁₋₆ alkanoyl, amino or C₁₋₆ acylamino;
pyrrolylcarbonyl optionally substituted with 1-3 of C₁₋₆ alkyl;
C₁₋₂ acyloxyacetyl where the acyl is optionally substituted with amino, C₁₋₆ alkylamino, C₁₋₆ dialkylamino, 4-morpholino, 4-aminophenyl, 4-(dialkylamino)phenyl, 4-(glycylamino)phenyl; or
R₅ and R₆ taken together with any intervening atoms can form a 3 to 7 membered heterocyclic ring containing carbon atoms and 1-2 heteroatoms independently chosen from O, S, SO, SO₂, N, or NR₈;
R₇ represent
hydrogen, halogen, CN, CO₂R, CON(R)₂, CHO, CH₂NHAc, C(═NOR), OH, C₁₋₆ alkoxy, C₁₋₆ alkyl, alkenyl,
(CH₂)ₙamino, (CH₂)ₙC1-6 alkylamino, C₁₋₆ dialkylamino, hydroxylamino or C₁₋₂ alkoxyamino all of which can be optionally substituted on the nitrogen with C₁₋₆ acyl, C₁₋₆ alkylsulfonyl or C₁₋₆ alkoxycarbonyl, said acyl and alkylsulfonyl optionally substituted with 1-2 of halogen or OH;
R₈ and R₉ independently represents
H, CN,
C₁₋₆ alkyl optionally substituted with 1-3 halogen, CN, OH, C₁₋₆ alkoxy, C₁₋₆ acyloxy, or amino,
phenyl optionally substituted with 1-3 groups of halogen, OH, C₁₋₆ alkoxy; or
R₇ and R₈ taken together can form a 3-7 membered carbon ring optionally interrupted with 1-2 heteroatoms chosen from O, S, SO, SO₂, NH, and NR₈;
X₁ represents O, S or NR₁₃, NCN, NCO₂R₁₆, or NSO₂R₁₄
R₁₀ represents hydrogen, C₁₋₆ alkyl or CO₂R₁₅;
Each R₁₃ represents independently hydrogen, C₁₋₆ alkyl, C₆₋₁₀ aryl, NR₅R₆, SR₈, S(O)R₈, S(O)₂R₈, CN, OH, C₁₋₆ alkylS(O)R, C₁₋₆ alkoxycarbonyl, hydroxycarbonyl, C₁₋₆ acyl, C₃₋₇ membered carbon ring optionally interrupted with 1-4 heteroatoms chosen from O, S, SO, SO₂, NH and NR₈ where said C₁₋₆ alkyl, aryl or C₁₋₆ acyl groups may be independently substituted with 0-3 halogens, hydroxy, N(R)₂, CO₂R, C₆₋₁₀ aryl, C₅₋₁₀ heteroaryl, or C₁₋₆ alkoxy groups;
When two R₁₃ groups are attached to the same atom or two adjacent atoms they may be taken together to form a 3-7 membered carbon ring optionally interrupted with 1-2 heteroatoms chosen from O, S, SO, SO₂, NH, and NR₈;
R represents hydrogen or C₁₋₆ alkyl;
R₁₄ represents amino, C₁₋₆ alkyl, C₁₋₆ haloalkyl, five to six membered heterocycles or phenyl, said phenyl and heterocycles optionally substituted with 1-3 group of halo, C₁₋₆ alkoxy, C₁₋₆ acylamino, or C₁₋₆ alkyl, hydroxy and/or amino, said amino and hydroxy optionally protected with an amino or hydroxy protecting group;
R₁₅ is C₁₋₆ alkyl or benzyl said benzyl optionally substituted with 1-3 groups of halo, OH, C₁₋₆ alkoxy, amino, C₁₋₆ acylamino, or C₁₋₆ alkyl;
R₁₆ is hydrogen, C₅₋₁₀heteroaryl, C₆₋₁₀aryl, said heteroaryl and aryl optionally substituted with 1-3 groups of R₇;
m, n, p and q represents 0-1.

Another aspect of the invention is concerned with the use of the novel antibiotic compositions in the treatment of bacterial infections.

### DETAILED DESCRIPTION OF THE INVENTION

The invention is described herein in detail using the terms defined below unless otherwise specified.

The compounds of the present invention may have asymmetric centers, chiral axes and chiral planes, and occur as racemates, racemic mixtures, and as individual diastereomers, with all possible isomers, including optical isomers, being included in the present invention. (See E. L. Eliel and S. H. Wilen Stereochemistry of Carbon Compounds (John Wiley and Sons, New York 1994, in particular pages 1119-1190).

When any variable (e.g. aryl, heterocycle, R₅, R₆ etc.) occurs more than once, its definition on each occurrence is independent at every other occurrence. Also combinations of substituents/or variables are permissible only if such combinations result in stable compounds.

The term “alkyl” refers to a monovalent alkane (hydrocarbon) derived radical containing from 1 to 15 carbon atoms unless otherwise defined. It may be straight or branched. Preferred alkyl groups include lower alkyls which have from 1 to 6 carbon atoms such as methyl, ethyl, propyl, isopropyl, butyl and t-butyl. When substituted, alkyl groups may be substituted with up to 3 substituent groups, selected from the groups as herein defined, at any available point of attachment. When the alkyl group is said to be substituted with an alkyl group, this is used interchangeably with “branched alkyl group”.

Cycloalkyl is a species of alkyl containing from 3 to 15 carbon atoms, without alternating or resonating double bonds between carbon atoms. It may contain from 1 to 4 rings which are fused. Preferred cycloalkyl groups are cyclopropyl, cyclobutyl, cyclopentyl and cyclohexyl. When substituted, cycloalkyl groups may be substituted with up to 3 substituents which are defined herein by the definition of alkyl.

Alkanoyl refers to a group derived from an aliphatic carboxylic acid of 2 to 4 carbon atoms. Examples are acetyl, propionyl, butyryl and the like.

The term “alkoxy” refers to those groups of the designated length in either a straight or branched configuration and if two or more carbon atoms in length, they may include a double or a triple bond. Exemplary of such alkoxy groups are methoxy, ethoxy, propoxy, isopropoxy, butoxy, isobutoxy, tertiary butoxy, pentoxy, isopentoxy, hexoxy, isohexoxy allyloxy, propargyloxy, and the like. 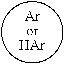
refers to aryl or heteroaryl, heterocycle, Het, heterocyclyl or heterocyclic as described immediately below.

Aryl refers to any stable monocyclic or bicyclic carbon ring of up to 7 atoms in each ring, wherein at least one ring is aromatic. Examples of such aryl elements include phenyl, napthyl, tetrahydronaphthyl, indanyl, indanonyl, biphenyl, tetralilnyl, tetralonyl, fluorenonyl, phenanthryl, anthryl, acenaphthyl, and the like substituted phenyl and the like. Aryl groups may likewise be substituted as defined. Preferred substituted aryls include phenyl and naphthyl.

The term heterocycle, heteroaryl, Het, heterocyclyl or heterocyclic, as used herein except where noted, represents a stable 5- to 7-membered mono- or bicyclic or stable 8- to 11-membered bicyclic heterocyclic ring system, any ring of which may be saturated or unsaturated, and which consists of carbon atoms and from one to four heteroatoms selected from the group consisting of N, O and S, and wherein the nitrogen and sulfur heteroatoms may optionally be oxidized, and the nitrogen heteroatom may optionally be quaternized (in which case it is properly balanced by a counterion), and including any bicyclic group in which any of the above-defined heterocyclic rings is fused to a benzene ring. The heterocyclic ring may be attached at any heteroatom or carbon atom, which results in the creation of a stable structure. The term heterocycle or heterocyclic includes heteroaryl moieties. “Heterocycle” or “heterocyclyl” therefore includes the above mentioned heteroaryls, as well as dihydro and tetrahydro analogs thereof. The heterocycle, heteroaryl, Het or heterocyclic may be substituted with 1-3 groups of R₇. Examples of such heterocyclic elements include, but are not limited to the following: piperidinyl, piperazinyl, 2-oxopiperazinyl, 2-oxopiperidinyl, 2-oxopyrrolodinyl, 2-oxazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyrimidonyl, pyridinonyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thiophenyl, imidazopyridinyl, triazolyl, tetrazolyl, triazinyl, thienyl, benzothienyl, thiamorpholinyl sulfoxide, thiamorpholinyl sulfone, naphthpyridinyl, dihydropyrimidinyl, dihydropyrrolyl, dihydroquinolinyl, dihydrotetrazolyl, dihydrotriazolyl, dihydrothienyl, dihydrooxazolyl, dihydrobenzothiophenyl, dihydrofuranyl, benzothiazolyl, benzothienyl, benzoimidazolyl, benzopyranyl, benzothiofuranyl, carbolinyl, chromanyl, cinnolinyl, benzopyrazolyl, benzodioxolyl and oxadiazolyl. Additional examples of heteroaryls are illustrated by formulas a, b, c and d: 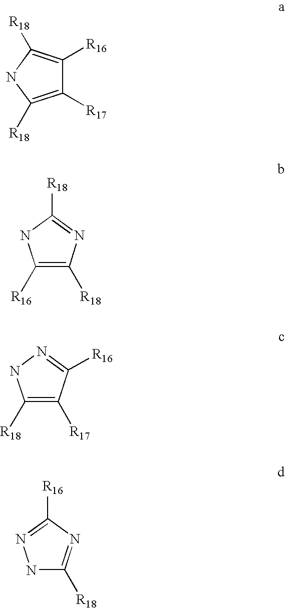
wherein R₁₆ and R₁₇ are independently selected from hydrogen, halogen, C₁₋₆ alkyl, C₂₋₄ alkanoyl, C₁₋₆ alkoxy; and R₁₈ represents hydrogen, C₁₋₆ alkyl, C₂₋₄ alkanoyl, C₁₋₆ alkoxycarbonyl and carbamoyl.

The expression 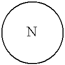
represents an optionally substituted aromatic heterocyclic group containing 1 to 4 nitrogen atoms and at least one double bond, and which is connected through a bond on any nitrogen and is optionally substituted with 1 to 3 groups of R₇. Exemplary groups are 1,2,3-triazole, 1,2,4-triazole, 1,2,5-triazole, tetrazole, pyrazole, and imidazole, any of which may contain 1 to 3 substitutents R₇.

The term “alkenyl” refers to a hydrocarbon radical straight, branched or cyclic containing from 2 to 10 carbon atoms and at least one carbon to carbon double bond. Preferred alkenyl groups include ethenyl, propenyl, butenyl and cyclohexenyl.

The terms “quaternary nitrogen” and “positive charge” refer to tetravalent, positively charged nitrogen atoms (balanced as needed by a counterion known in the art) including, e.g., the positively charged nitrogen in a tetraalkylammonium group (e.g. tetramethylammonium), heteroarylium, (e.g., N-methyl-pyridinium), basic nitrogens which are protonated at physiological pH, and the like. Cationic groups thus encompass positively charged nitrogen-containing groups, as well as basic nitrogens which are protonated at physiologic pH.

The term “heteroatom” means O, S or N, selected on an independent basis.

The term “prodrug” refers to compounds which are drug precursors which, following administration and absorption, release the drug in vivo via some metabolic process. Exemplary prodrugs include acyl amides of the amino compounds of this invention such as amides of alkanoic(C₁₋₆)acids, amides of aryl acids (e.g., benzoic acid) and alkane(C₁₋₆)dioic acids.

Halogen and “halo” refer to bromine, chlorine, fluorine and iodine.

When a group is termed “substituted”, unless otherwise indicated, this means that the group contains from 1 to 3 substituents thereon.

When a functional group is termed “protected”, this means that the group is in modified form to preclude undesired side reactions at the protected site. Suitable protecting groups for the compounds of the present invention will be recognized from the present application taking into account the level of skill in the art, and with reference to standard textbooks, such as Greene, T. W. et al. Protective Groups in Organic Synthesis Wiley, New York (1991). Examples of suitable protecting groups are contained throughout the specification.

Examples of suitable hydroxyl and amino protecting groups are: trimethylsilyl, triethylsilyl, o-nitrobenzyloxycarbonyl, p-nitrobenzyloxycarbonyl, t-butyldiphenylsilyl, t-butyldimethylsilyl, benzyloxycarbonyl, t-butyloxycarbonyl, 2,2,2-trichloroethyloxycarbonyl, allyloxycarbonyl and the like. Examples of suitable carboxyl protecting groups are benzhydryl, o-nitrobenzyl, p-nitrobenzyl, 2-naphthylmethyl, allyl, 2-chloroallyl, benzyl, 2,2,2-trichloroethyl, trimethylsilyl, t-butyldimethylsilyl, t-butyldiphenylsilyl, 2-(trimethylsilyl)ethyl, phenacyl, p-methoxybenzyl, acetonyl, p-methoxyphenyl, 4-pyridylmethyl, t-butyl and the like.

The cyclopropyl containing oxazolidinone compounds of the present invention are useful per se and in their pharmaceutically acceptable salt and ester forms for the treatment of bacterial infections in animal and human subjects. The term “pharmaceutically acceptable ester, salt or hydrate,” refers to those salts, esters and hydrated forms of the compounds of the present invention which would be apparent to the pharmaceutical chemist. i.e., those which are substantially non-toxic and which may favorably affect the pharmacokinetic properties of said compounds, such as palatability, absorption, distribution, metabolism and excretion. Other factors, more practical in nature, which are also important in the selection, are cost of the raw materials, ease of crystallization, yield, stability, solubility, hygroscopicity and flowability of the resulting bulk drug. Conveniently, pharmaceutical compositions may be prepared from the active ingredients in combination with pharmaceutically acceptable carriers. Thus, the present invention is also concerned with pharmaceutical compositions and methods of treating bacterial infections utilizing as an active ingredient the novel cyclopropyl containing oxazolidinone compounds.

The pharmaceutically acceptable salts referred to above also include acid addition salts. Thus, when the Formula I compounds are basic, salts may be prepared from pharmaceutically acceptable non-toxic acids, including inorganic or organic acids. Included among such acid salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, isethionic, lactate, maleate, mandelic, malic, maleic, methanesulfonate, mucic, 2-naphthalenesulfonate, nicotinate, nitric oxalate, pamoate, pectinate, persulfate, 3-phenylpropionate, picrate, pivalate, propionate, phosphate, pantothenic, pamoic, sulfate, succinate, tartrate, thiocyanate, tosylate and undecanoate.

When the compound of the present invention is acidic, suitable “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium zinc and the like. Particularly preferred are the ammonium, calcium, magnesium, potassium and sodium salts. Salts derived from pharmaceutically acceptable inorganic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

The pharmaceutically acceptable esters are such as would be readily apparent to a medicinal chemist, and include those which are hydrolyzed under physiological conditions, such as “biolabile esters”, pivaloyloxymethyl, acetoxymethyl, phthalidyl, indanyl and methoxymethyl, and others.

Biolabile esters are biologically hydrolizable, and may be suitable for oral administration, due to good absorption through the stomach or intestinal mucosa, resistance to gastric acid degrada-tion and other factors. Examples of biolabile esters include compounds.

An embodiment of this invention is realized when R₁ and R₂ independently represent H, NR₅R₆, CN, OH, C(R)₂OR₁₄, NHC(═X₁)N(R₁₃)₂, C(═NOH)N(R₁₃)₂, NR₁₀C(═X₁)R₁₃ or CR₇R₈R₉ and all other variables are as described herein.

Another embodiment of this invention is realized when 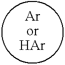
is phenyl, pyridine, pyrimidine, or piperidine and all other variables are as described herein.

Another embodiment of this invention is realized when one of R₁ and R₂ is H and the other is NR₅R₆ and all other variables are as described herein.

Another embodiment of this invention is realized when one of R₁ and R₂ is H and the other is CN and all other variables are as described herein.

Another embodiment of this invention is realized when one of R₁ and R₂ is H and the other is NR₁₀C(═X₁)R₁₃ and all other variables are as described herein.

Another sub-embodiment of this invention is realized when A is N, [symbol] is not present, Z=(O)ₙ where n=1 and all other variables are as described herein.

Another sub-embodiment of this invention is realized when A is C, [symbol] is present and Z=(O)ₙ where n=0 and all other variables are as described herein.

Another sub-embodiment of this invention is realized when A is C, [symbol] is not present and Z=H, OH or halogen and all other variables are as described herein.

Another embodiment of this invention is realized when R₃ is 1,2,3-triazol-1-yl optionally substituted with 1-3 groups of Ra and all other variables are as described herein.

Still another embodiment of this invention is realized when R₅ and R₆ independently are:

H,

C₁₋₆ alkyl optionally substituted with 1-3 groups of halogen, CN, OH, C₁₋₆ alkoxy, amino, hydroxyamino, alkoxyamino, C₁₋₆ acyloxy, C₁₋₆ alkylsulfenyl, C₁₋₆ alkylsulfinyl, C₁₋₆ alkylsulfonyl, aminosulfonyl, C₁₋₆ alkylaminosulfonyl, C₁₋₆ dialkylaminosulfonyl, 4-morpholinylsulfonyl, phenyl, pyridine, 5-isoxazolyl, ethylenyloxy, or ethynyl, said phenyl and pyridine optionally substituted with 1-3 halogen, CN, OH, CF₃, C₁₋₆ alkyl or C₁₋₆ alkoxy;

C₁₋₆ acyl optionally substituted with 1-3 groups of halogen, OH, SH, C₁₋₆ alkoxy, naphthaleneoxy, phenoxy, amino, C₁₋₆ acylamino, hydroxylamino, alkoxylamino, C₁₋₆ acyloxy, phenyl, pyridine, C₁₋₆ alkylcarbonyl, C₁₋₆ alkylamino, C₁₋₆ dialkylamino, C₁₋₆ hydroxyacyloxy, C₁₋₆ alkylsulfenyl, phthalimido, maleimido, succinimido, said phenoxy, phenyl and pyridine optionally substituted with 1-3 groups of halo, OH, CN, C₁₋₆ alkoxy, amino, C₁₋₆ acylamino, CF₃ or C₁₋₆ alkyl; or

benzoyl optionally substituted with 1-3 groups of halogen, OH, C₁₋₆ alkoxy, C₁₋₆ alkyl, CF₃, C₁₋₆ alkanoyl, amino or C₁₋₆ acylamino and all other variables are as described herein.

Yet another embodiment of this invention is realized when X₁ represents O and all other variables are as described herein.

Preferred compounds of this invention are:
1-[5(R)-3-[4-[(1α,5α,6α)-6-amino-3-azabicyclo[3.1.0]hexan-3-yl]-3-fluorophenyl]-2-oxooxazolidin-5-ylmethyl]-1,2,3-triazole,
1-[5(R)-3-[4-[(1α,5α,6α)-6-amino-3-azabicyclo[3.1.0]hexan-3-yl]-3,5-difluorophenyl]-2-oxooxazolidin-5-ylmethyl]-1,2,3-triazole,
1-[5(R)-3-[4-[(1α,5α,6α)-6-[(t-butyldiphenylsilyl)oxy]methylbicyclo[3.1.0]hex-2-en-3-yl]phenyl]-2-oxooxazolidin-5-ylmethyl]-1,2,3-triazole,
1-[5(R)-3-[4-[(1α,5α,6α)-6-[(t-butyldiphenylsilyl)oxy]methylbicyclo[3.1.0]hex-2-en-3-yl]-3-fluorophenyl]-2-oxooxazolidin-5-ylmethyl]-1,2,3-triazole,
1-[5(R)-3-[4-[(1α,5α,6α)-6-hydroxyoxymethylbicyclo[3.1.0]hex-2-en-3-yl]phenyl]-2-oxooxazolidin-5-ylmethyl]-1,2,3-triazole,
1-[5(R)-3-[3-fluoro-4-[(1α,5α,6α)-6-hydroxyoxymethylbicyclo[3.1.0]hex-2-en-3-yl]phenyl]-2-oxooxazolidin-5-ylmethyl]-1,2,3-triazole,
1-[5(R)-3-[4-[(1α,5α,6α)-6-cyanobicyclo[3.1.0]hex-2-en-3-yl]phenyl]-2-oxooxazolidin-5-ylmethyl]-1,2,3-triazole,
1-[5(R)-3-[4-[(1α,5α,6α)-6-Cyanobicyclo[3.1.0]hex-2-en-3-yl]-3-fluorophenyl]-2-oxooxazolidin-5-ylmethyl]-1,2,3-triazole,
   or their enantiomer, diastereomer, or pharmaceutically acceptable salt, hydrate or prodrug thereof wherein.

Suitable subjects for the administration of the formulation of the present invention include mammals, primates, man, and other animals. In vitro antibacterial activity is predictive of in vivo activity when the compositions are administered to a mammal infected with a susceptible bacterial organism.

Using standard susceptibility tests, the compositions of the invention are determined to be active against MRSA and enterococcal infections.

The compounds of the invention are formulated in pharmaceutical compositions by combining the compounds with a pharmaceutically acceptable carrier. Examples of such carriers are set forth below.

The compounds may be employed in powder or crystalline form, in liquid solution, or in suspension. They may be administered by a variety of means; those of principal interest include: topically, orally and parenterally by injection (intravenously or intramuscularly).

Compositions for injection, a preferred route of delivery, may be prepared in unit dosage form in ampules, or in multidose containers. The injectable compositions may take such forms as suspensions, solutions, or emulsions in oily or aqueous vehicles, and may contain various formulating agents. Alternatively, the active ingredient may be in powder (lyophilized or non-lyophilized) form for reconstitution at the time of delivery with a suitable vehicle, such as sterile water. In injectable compositions, the carrier is typically comprised of sterile water, saline or another injectable liquid, e.g., peanut oil for intramuscular injections. Also, various buffering agents, preservatives and the like can be included.

Topical applications may be formulated in carriers such as hydrophobic or hydrophilic bases to form ointments, creams, lotions, in aqueous, oleaginous or alcoholic liquids to form paints or in dry diluents to form powders.

Oral compositions may take such forms as tablets, capsules, oral suspensions and oral solutions. The oral compositions may utilize carriers such as conventional formulating agents, and may include sustained release properties as well as rapid delivery forms.

The dosage to be administered depends to a large extent upon the condition and size of the subject being treated, the route and frequency of administration, the sensitivity of the pathogen to the particular compound selected, the virulence of the infection and other factors. Such matters, however, are left to the routine discretion of the physician according to principles of treatment well known in the antibacterial arts. Another factor influencing the precise dosage regimen, apart from the nature of the infection and peculiar identity of the individual being treated, is the molecular weight of the compound.

The novel antibiotic compositions of this invention for human delivery per unit dosage, whether liquid or solid, comprise from about 0.01% to as high as about 99% of the cyclopropyl containing oxazolidinone compounds discussed herein, the preferred range being from about 10-60% and from about 1% to about 99.99% of one or more of other antibiotics such as those discussed herein, preferably from about 40% to about 90%. The composition will generally contain from about 125 mg to about 3.0 g of the cyclopropyl containing oxazolidinone compounds discussed herein; however, in general, it is preferable to employ dosage amounts in the range of from about 250 mg to 1000 mg and from about 200 mg to about 5 g of the other antibiotics discussed herein; preferably from about 250 mg to about 1000 mg. In parenteral administration, the unit dosage will typically include the pure compound in sterile water solution or in the form of a soluble powder intended for solution, which can be adjusted to neutral pH and isotonic.

The invention described herein also includes a method of treating a bacterial infection in a mammal in need of such treatment comprising administering to said mammal the claimed composition in an amount effective to treat said infection.

Oxazolidinones have been known at times to cause side effects such as sideroblastic anemia, peripheral sensory neuropathy, optic neuropathy, seizures, thrombocytopenia, cheilosis, seborrheic dermatitis, hypo-regenerative anemia, megaloblastic anemia or normocytic anemia. The compounds of the invention may be combined with an effective amount of one or more vitamins to prevent or reduce the occurrence of oxazolidinone-associated side effects in patients. The vitamins that can be combined are vitamin B2, vitamin B6, vitamin B12 and folic acid. The vitamins may be administered with the oxazolidinones as separate compositions or the vitamins and oxazolidinones may be present in the same composition.

Thus another aspect of this invention is a method of treating or preventing an oxazolidinone-associated side effect by administering an effective amount of the oxazolidinone of structural formula I and an effective amount of one or more of vitamin B2, vitamin B6, vitamin B12 and folic acid to a patient in need thereof.

A further aspect of this invention relates to a method of treating or preventing oxazolidinone-associated normocytic anemia or peripheral sensory neuropathy by administering an effective amount of vitamin B2 to a patient in need thereof.

Yet another aspect of this invention relates to a method of treating or preventing oxazolidinone-associated sideroblastic anemia, peripheral sensory neuropathy, optic neuropathy, seizures, thrombocytopenia, cheilosis, and seborrheic dermatitis by administering an effective amount of vitamin B6 to a patient in need thereof.

Still another aspect of this invention relates to a method of treating or preventing oxazolidinone-associated hypo-regenerative anemia, megaloblastic anemia by administering an effective amount of vitamin B12 and folic acid to a patient in need thereof.

Still another aspect of this invention relates to a method of treating or preventing bacterial infection by administering an effective amount of a compound of formula I and an effective amount of one or more of the group selected from the group consisting of vitamin B2, vitamin B6, vitamin B12 and folic acid to a patient in need thereof.

The preferred methods of administration of the claimed compositions include oral and parenteral, e.g., i.v. infusion, i.v. bolus and i.m. injection formulated so that a unit dosage comprises a therapeutically effective amount of each active component or some submultiple thereof.

For adults, about 5-50 mg/kg of body weights preferably about 250 mg to about 1000 mg per person of the cyclopropyl containing oxazolidinone antibacterial compound and about 250 mg, to about 1000 mg per person of the other antibiotic(s) given one to four times daily is preferred. More specifically, for mild infections a dose of about 250 mg two or three times daily of the cyclopropyl containing oxazolidinone antibacterial compound and about 250 mg two or three times daily of the other antibiotic is recommended. For moderate infections against highly susceptible gram positive organisms a dose of about 500 mg each of the cyclopropyl containing oxazolidinone and the other antibiotics, three or four times daily is recommended. For severe, life-threatening infections against organisms at the upper limits of sensitivity to the antibiotic, a dose of about 500-2000 mg each of the cyclopropyl-containing oxazolidinone compound and the other antibiotics, three to four times daily may be recommended.

For children, a dose of about 5-25 mg/kg of body weight given 2, 3, or 4 times per day is preferred; a dose of 10 mg/kg is typically recommended.

The invention is further described in connection with the following non-limiting examples.

### EXAMPLE 1

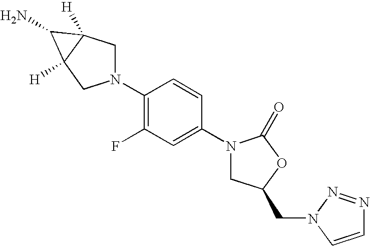

### 1-[5(R)-3-[4-[(1α,5α,6α)-6-Amino-3-azabicyclo[3.1.0]hexan-3-yl]-3-fluorophenyl]-2-oxooxazolidin-5-ylmethyl]-1,2,3-triazole

Step 1.

### 1-[5(R)-3-[4-[(1α,5α,6α)-6-(N-t-Butoxycarbonyl)amino-3-azabicyclo[3.1.0]hexan-3-yl]-3-fluorophenyl]-2-oxooxazolidin-5-ylmethyl]-1,2,3-triazole

The mixture of 1-[5(R)-azidomethyl-3-[4-[(1α,5α,6α)-6-(N-t-butoxycarbonyl)amino-3-azabicyclo[3.1.0]hexan-3-yl]-3-fluorophenyl]oxazolidin-2-one (370 mg) and 2,5-norbornadiene (891 mg) in dioxane (6.5 mL) was heated at 70° C. for 6 hours, and then concentrated in vacuo. A suspension of the residue in diethyleneglycol dimethylether (13.5 μL) was heated at 140° C. for 10 minutes, and then concentrated in vacuo. Flash chromatography (silica, dichloromethane:methanol=20:1) of the residue gave 1-[5(R)-3-[4-[(1α,5α,6α)-6-(N-t-butoxycarbonyl)amino-3-azabicyclo[3.1.0]hexan-3-yl]-3-fluorophenyl]-2-oxooxazolidin-5-ylmethyl]-1,2,3-triazole (261 mg).

MS (EI⁺) m/z: 458 (M⁺).

HRMS (EI⁺) for C₂₂H₂₇FN₆O₄ (M⁺): calcd, 458.2078; found, 458.2072.

Step 2.

### 1-[5(R)-3-[4-[(1α,5α,6α)-6-Amino-3-azabicyclo[3.1.0]hexan-3-yl]-3-fluorophenyl]-2-oxooxazolidin-5-ylmethyl]-1,2,3-triazole

To a solution of 1-[5(R)-3-[4-[(1α,5α,6α)-6-(N-t-butoxycarbonyl)amino-3-azabicyclo[3.1.0]hexan-3-yl]-3-fluorophenyl]-2-oxooxazolidin-5-ylmethyl]-1,2,3-triazole (291 mg) in methanol (12 mL) was added a solution of 12 N hydrochloric acid in methanol (0.79 mL), the mixture was stirred at room temperature for 10.5 hours, and then concentrated in vacuo. The mixture was diluted with dichloromethane, and extracted with 1 N hydrochloric acid solution. The aqueous extracts were made to alkaline by the addition of sodium hydrogencarbonate and sodium carbonate. The resulting mixture was extracted with dichloromethane. The organic extracts were dried over anhydrous sodium sulfate, filtered, and then concentrated in vacuo. Flash chromatography (NH silica, dichloromethane:methanol=20:1) of the residue gave 1-[5(R)-3-[4-[(1α,5α,6α)-6-amino-3-azabicyclo[3.1.0]hexan-3-yl]-3-fluorophenyl]-2-oxooxazolidin-5-ylmethyl]-1,2,3-triazole (224 mg).

MS (FAB⁺) m/z: 359 (MH⁺).

HRMS (FAB⁺) for C₁₇H₂₀FN₆O₂ (MH⁺): calcd, 359.1632; found, 359.1646.

### EXAMPLE 2

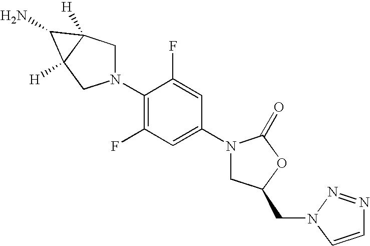

### 1-[5(R)-3-[4-[(1α,5α,6α)-6-Amino-3-azabicyclo[3.1.0]hexan-3-yl]-3,5-difluorophenyl]-2-oxooxazolidin-5-ylmethyl]-1,2,3-triazole

Step 1.

### 1-[5(R)-3-[4-[(1α,5α,6α)-6-(N-t-Butoxycarbonyl)amino-3-azabicyclo[3.1.0]hexan-3-yl]-3,5-difluorophenyl]-2-oxooxazolidin-5-ylmethyl]-1,2,3-triazole

The title compound 1-[5(R)-3-[4-[(1α,5α,6α)-6-(N-t-butoxycarbonyl)amino-3-azabicyclo[3.1.0]hexan-3-yl]-3,5-difluorophenyl]-2-oxooxazolidin-5-ylmethyl]-1,2,3-triazole (339 mg) was prepared from 5(R)-azidomethyl-3-[4-[(1α,5α,6α)-6-(N-t-butoxycarbonyl)amino-3-azabicyclo[3.1.0]hexan-3-yl]-3,5-difluorophenyl]oxazolidin-2-one (450 mg) and 2,5-norbornadiene (1.04 g) in the same manner as described for EXAMPLE 1.

MS (EI⁺) m/z: 476 (M⁺).

HRMS (EI⁺) for C₂₂H₂₆F₂N₆O₄ (M⁺): calcd, 476.1984; found, 476.2008.

Step 2.

### 1-[5(R)-3-[4-[(1α,5α,6α)-6-Amino-3-azabicyclo[3.1.0]hexan-3-yl]-3,5-difluorophenyl]-2-oxooxazolidin-5-ylmethyl]-1,2,3-triazole

The title compound 1-[5(R)-3-[4-[(1α,5α,6α)-6-amino-3-azabicyclo[3.1.0]hexan-3-yl]-3,5-difluorophenyl]-2-oxooxazolidin-5-ylmethyl]-1,2,3-triazole (251 mg) was prepared from 1-[5(R)-3-[4-[(1α,5α,6α)-6-(N-t-butoxycarbonyl)amino-3-azabicyclo[3.1.0]hexan-3-yl]-3,5-difluorophenyl]-2-oxooxazolidin-5-ylmethyl]-1,2,3-triazole (339 mg) in the same manner as described for EXAMPLE 1.

MS (FAB⁺) m/z: 377 (H).

HRMS (FAB⁺) for C₁₇H₁₉F₂N₆O₂ (MH⁺): calcd, 377.1538; found, 377.1526.

### EXAMPLE 3

### 1-[5(R)-3-[4-[(1α,5α,6α)-6-[(t-Butyldiphenylsilyl)oxy]methylbicyclo[3.1.0]hex-2-en-3-yl]phenyl]-2-oxooxazolidin-5-ylmethyl]-1,2,3-triazole

To a suspension of 1-[5(R)-3-(4-iodophenyl)-2-oxooxazolidin-5-ylmethyl]-1,2,3-triazole (1.56 g), (1α,5α,6α)-6-[(t-butyldiphenylsilyl)oxy]methyl-3-(4,4,5,5-tetramethyl-1, 3,2-dioxaborolyl)bicyclo[3.1.0]hex-2-ene (1.90 g) and tetrakis(triphenylphosphine)palladium-(0) (477 mg) in dioxane (100 mL) was added a solution of 1 M tri-potassium phosphate solution (20 mL), the mixture was stirred at 80° C. for 2 hours. After dilution of the mixture with water and ethyl acetate, the insoluble materials were filtered off, and the filtrate was extracted with ethyl acetate. The organic extracts were dried over anhydrous sodium sulfate, and then concentrated in vacuo. Flash chromatography (silica, hexane:ethyl acetate=1:10) of the residue gave 1-[5(R)-3-[4-[(1α,5α,6α)-6-[(t-butyldiphenylsilyl)oxy]methylbicyclo[3.1.0]hex-2-en-3-yl]phenyl]-2-oxooxazolidin-5-ylmethyl]-1,2,3-triazole (1.85 g).

MS (FAB⁺) m/z: 591 (MH⁺).

HRMS (FAB⁺) for C₃₅H₃₉N₄O₃Si(MH⁺): calcd, 591.2791; found, 591.2770.

### EXAMPLE 4

### 1-[5(R)-3-[4-[(1α,5α,6α)-6-[(t-Butyldiphenylsilyl)oxy]methylbicyclo[3.1.0]hex-2-en-3-yl]-3-fluorophenyl]-2-oxooxazolidin-5-ylmethyl]-1,2,3-triazole

The title compound 1-[5(R)-3-[4-[(1α,5α,6α)-6-[(t-butyldiphenylsilyl)oxy]methylbicyclo[3.1.0]hex-2-en-3-yl]-3-fluorophenyl]-2-oxooxazolidin-5-ylmethyl]-1,2,3-triazole (2.32 g) was prepared from 1-[5(R)-3-(3-fluoro-4-iodophenyl)-2-oxooxazolidin-5-ylmethyl]-1,2,3-triazole (1.63 g) and (1α,5α,6α)-6-[(t-butyldiphenylsilyl)oxy]methyl-3-(4,4,5,5-tetramethyl-1,3,2-dioxaborolyl)bicyclo[3.1.0]hex-2-ene (1.90 g) in the same manner as described for EXAMPLE 3.

MS (FAB⁺) m/z: 609 (MH⁺).

HRMS (FAB⁺) for C₃₅H₃₈FN₄O₃Si (MH⁺): calcd, 609.2697; found, 609.2689.

### EXAMPLE 5

### 1-[5(R)-3-[4-[(1α,5α,6α)-6-Hydroxyoxymethylbicyclo[3.1.0]hex-2-en-3-yl]phenyl]-2-oxooxazolidin-5-ylmethyl]-1,2,3-triazole

To a solution of 1-[5(R)-3-[4-[(1α,5α,6α)-6-[(t-butyldiphenylsilyl)oxy]methylbicyclo[3.1.0]hex-2-en-3-yl]phenyl]-2-oxooxazolidin-5-ylmethyl]-1,2,3-triazole (1.85 g) in tetrahydrofuran (6.3 mL) was added a solution of tetrabutylammonium fluoride in tetrahydrofuran (1 M, 6.3 mL) at 0° C., the mixture was stirred at room temperature overnight. Flash chromatography (silica, ethyl acetate:methanol=10:1) of the residue gave 1-[5(R)-3-[4-[(1α,5α,6α)-6-hydroxyoxymethylbicyclo[3.1.0]hex-2-en-3-yl]phenyl]-2-oxooxazolidin-5-ylmethyl]-1,2,3-triazole (914 mg).

MS (EI⁺) m/z: 352 (M⁺).

HRMS (EI⁺) for C₁₉H₂₀N₄O₃ (M⁺): calcd, 352.1535; found, 352.1573.

### EXAMPLE 6

### 1-[5(R)-3-[3-Fluoro-4-[(1α,5α,6α)-6-hydroxyoxymethylbicyclo[3.1.0]hex-2-en-3-yl]phenyl]-2-oxooxazolidin-5-ylmethyl]-1,2,3-triazole

The title compound 1-[5(R)-3-[3-fluoro-4-[(1α,5α,6α)-6-hydroxyoxymethylbicyclo[3.1.0]hex-2-en-3-yl]phenyl]-2-oxooxazolidin-5-ylmethyl]-1,2,3-triazole (997 mg) was prepared from 1-[5(R)-3-[4-[(1α,5α,6α)-6-[(t-butyldiphenylsilyl)oxy]methylbicyclo[3.1.0]hex-2-en-3-yl]-3-fluorophenyl]-2-oxooxazolidin-5-ylmethyl]-1,2,3-triazole (2.32 g) in the same manner as described for EXAMPLE 5.

MS (EI⁺) m/z: 370 (M).

HRMS (EI⁺) for C₁₉H₁₉FN₄O₃ (M⁺): calcd, 370.1441; found, 370.1443.

### EXAMPLE 7

### 1-[5(R)-3-[4-[(1α,5α,6α)-6-Cyanobicyclo[3.1.0]hex-2-en-3-yl]phenyl]-2-oxooxazolidin-5-ylmethyl]-1,2,3-triazole

To a suspension of 1-[5(R)-3-[4-[(1α,5α,6α)-6-hydroxymethylbicyclo[3.1.0]hex-2-en-3-yl]phenyl]-2-oxooxazolidin-5-ylmethyl]-1,2,3-triazole (599 mg), N-methylmorpholine N-oxide (308 mg) and molecular sieves 4A (powdered, 850 mg) in dichloromethane (34 mL) and acetonitrile (3.4 mL) was added tetrapropylammonium perruthenate (67.7 mg) at room temperature, the resulting mixture was stirred for 6 hours. After insoluble materials were filtered off, the filtrate was concentrated in vacuo to give 1-[5(R)-3-[3-fluoro-4-[(1α,5α,6α)-6-formylbicyclo[3.1.0]hex-2-en-3-yl]phenyl]-2-oxooxazolidin-5-ylmethyl]-1,2,3-triazole. This was used in the next step without further purification. To a suspension of the residue in methanol (17 mL) was added N,N-dimethylhydrazine (1.5 mL) at room temperature, the mixture was stirred at 40° C. for 6 hours, and then concentrated in vacuo. To a suspension of the residue in methanol (17 mL) was added magnesium monoperoxyphthalate hexahydrate (2.10 g) at 0° C., the mixture was stirred at the same temperature for 20 minutes. After dilution of the mixture with water, the resulting precipitates were collected by filtration to give 1-[5(R)-3-[4-[(1α,5α,6α)-6-cyanobicyclo[3.1.0]hex-2-en-3-yl]phenyl]-2-oxooxazolidin-5-ylmethyl]-1,2,3-triazole (334 mg).

MS (FAB⁺) m/z: 348 (MH⁺).

HRMS (FAB⁺) for C₁₉H₁₈N₅O₂ (MH⁺): calcd, 348.1460; found, 348.1480.

### EXAMPLE 8

### 1-[5(R)-3-[4-[(1α,5α,6α)-6-Cyanobicyclo[3.1.0]hex-2-en-3-yl]-3-fluorophenyl]-2-oxooxazolidin-5-ylmethyl]-1,2,3-triazole

The title compound 1-[5(R)-3-[4-[(1α,5α,6α)-6-cyanobicyclo[3.1.0]hex-2-en-3-yl]-3-fluorophenyl]-2-oxooxazolidin-5-ylmethyl]-1,2,3-triazole (230 mg) was prepared from 1-[5(R)-3-[3-fluoro-4-[(1α,5α,6α)-6-hydroxymethylbicyclo[3.1.0]hex-2-en-3-yl]phenyl]-2-oxooxazolidin-5-ylmethyl]-1,2,3-triazole (630 mg) in the same manner as described for EXAMPLE 7.

MS (FAB⁺) m/z: 366 (H).

HRMS (FAB⁺) for C₁₉H₁₇FN₅O₂ (MH⁺): calcd, 366.1366; found, 366.1330.

### REFERENCE EXAMPLE 1

### 5(R)-3-[4-[(1α,5α,6α)-6-(N-Benzyl-N-t-butoxycarbonyl)amino-3-azabicyclo[3.1.0]hexan-3-yl]-3-fluorophenyl]-5-hydroxymethyloxazolidin-2-one

Step 1.

### 4-[(1α,5α,6α)-6-(N-t-Butoxycarbonyl)amino-3-azabicyclo[3.1.0]hexan-3-yl]-3-fluoronitrobenzene

To a suspension of (1α,5α,6α)-6-(N-t-butoxycarbonyl)amino-3-azabicyclo[3.1.0]hexane (2.97 g) and ethyldiisopropylamine (2.87 mL) in acetonitrile (17 mL) was added 3,4-difluoronitrobenzene (1.66 mL), and the mixture was stirred at 50° C. for 4.5 hours. After cooling, the resulting precipitates were collected by filtration, and then dried in vacuo to give 4-[(1α,5α,6α)-6-(N-t-butoxycarbonyl)amino-3-azabicyclo[3.1.0]hexan-3-yl]-3-fluoronitrobenzene (2.81 g). The filtrate was concentrated in vacuo, the residue was dissolved in ethyl acetate, washed with 1N hydrochloric acid, water, aqueous sodium hydrogencarbonate solution and brine, successively. The organic extracts were dried over anhydrous sodium sulfate, and then concentrated in vacuo. The residue was treated with hexane and ethyl acetate, and the resulting precipitates were collected by filtration, and then dried in vacuo to give the additional product (1.38 g). The filtrate was concentrated in vacuo. Flash chromatography (silica, hexane:ethyl acetate=10:7) of the residue gave the additional product (228 mg).

¹H NMR (CDCl₃) δ 1.46 (s, 9H), 1.90 (s, 2H), 2.41 (s, 1H), 3.63 (d, J=9.5 Hz, 2H), 3.92 (d, J=9.5 Hz, 2H), 6.52 (t, J=9.0 Hz, 1H), 7.85 (dd, J=14.2, 2.4 Hz, 1H), 7.91 (dd, J=9.0, 2.4 Hz, 1H).

MS (FAB⁺) m/z: 338 (MH⁺).

Step 2.

### 4-[(1α,5α,6α)-6-(N-Benzyl-M-t-butoxycarbonyl)amino-3-azabicyclo[3.1.0]hexan-3-yl]-3-fluoronitrobenzene

To a solution of 4-[(1α,5α,6α)-6-(N-t-butoxycarbonyl)amino-3-azabicyclo[3.1.0]hexan-3-yl]-3-fluoronitrobenzene in N,N-dimethylformamide (89 mL) was added sodium hydride (689 mg), and the mixture was stirred at room temperature for 20 min. and then stirred at 40° C. for 5 min. To the resulting solution were added benzyl chloride (1.75 mL) and tetrabutylammonium bromide (42.7 mg), and the mixture was stirred at 50° C. for 1 hour, and then concentrated in vacuo. The residue was dissolved in ethyl acetate, washed with water and brine, dried over anhydrous sodium sulfate, and then concentrated in vacuo. Flash chromatography (silica, hexane:ethyl acetate=5:2) of the residue gave 4-[(1α,5α,6α)-6-(N-benzyl-N-t-butoxycarbonyl)amino-3-azabicyclo[3.1.0]hexan-3-yl]-3-fluoronitrobenzene (5.19 g).

¹H NMR (CDCl₃) δ 1.49 (s, 9H), 2.01 (s, 2H), 2.27 (s, 1H), 3.62 (d, J=9.3 Hz, 2H), 3.80-3.90 (m, 2H), 4.46 (s, 2H), 6.46 (t, J=9.0 Hz, 1H), 7.20-7.40 (m, 5H), 7.83 (dd, J=14.4, 2.7 Hz, 1H), 7.89 (dd, J=9.0, 2.7 Hz, 1H).

MS (FAB⁺) m/z: 428 (MH⁺).

Step 3.

### 4-[(1α,5α,6α)-6-(N-Benzyl-N-t-butoxycarbonyl)amino-3-azabicyclo[3.1.0]hexan-3-yl]-1-benzyloxycarbonylamino-3-fluorobenzene

A suspension of 4-[(1α,5α,6α)-6-(N-benzyl-N-t-butoxycarbonyl)amino-3-azabicyclo[3.1.0]hexan-3-yl]-3-fluoronitrobenzene (5.19 g) and palladium catalyst (10% on charcoal, 519 mg) in ethyl acetate (52 mL) was hydrogenated at 1 atm for 2 hours at room temperature. After filtration of the catalyst, the filtrate was concentrated in vacuo to give 1-amino-4-[(1α,5α,6α)-6-(N-benzyl-N-t-butoxycarbonyl)amino-3-azabicyclo[3.1.0]hexan-3-yl]-3-fluorobenzene. This was used in the next step without further purification. To a solution of crude 1-amino-4-[(1α,5α,6α)-6-(N-benzyl-N-t-butoxycarbonyl)amino-3-azabicyclo[3.1.0]hexan-3-yl]-3-fluorobenzene thus obtained in acetone (48 mL) were successively added sodium hydrogencarbonate (1.12 g), water (11 mL) and benzyl chloroformate (2.01 mL) at 0° C., and the mixture was stirred at 0° C. for 15 min. The mixture was diluted with ethyl acetate, washed with brine. The organic extracts were dried over anhydrous sodium sulfate, filtered, and then concentrated in vacuo. Flash chromatography (silica, hexane:ethyl acetate=5:2) of the residue gave 4-[(1α,5α,6α)-6-(N-benzyl-N-t-butoxycarbonyl)amino-3-azabicyclo[3.1.0]hexan-3-yl]-1-benzyloxycarbonylamino-3-fluorobenzene (6.73 g).

¹H NMR (CDCl₃) δ 1.49 (s, 9H), 1.80-1.90 (m, 2H), 2.40-2.60 (m, 1H), 3.24 (d, J=8.5 Hz, 2H), 3.50-3.80 (m, 2H), 4.45 (s, 2H), 5.17 (s, 2H), 6.40-6.60 (m, 1H), 6.80-6.90 (m, 1H), 7.10-7.50 (m, 11H).

MS (EI⁺) m/z: 531 (M⁺).

Step 4.

### 5(R)-3-[4-[(1α,5α,6α)-6-N-Benzyl-N-t-butoxycarbonyl)amino-3-azabicyclo[3.1.0]hexan-3-yl]-3-fluorophenyl]-5-hydroxymethyloxazolidin-2-one

To a solution of 4-[(1α,5α,6α)-6-N-benzyl-N-t-butoxycarbonyl)amino-3-azabicyclo[3.1.0]hexan-3-yl]-1-benzyloxycarbonylamino-3-fluorobenzene (6.46 g) in dry tetrahydrofuran (65 mL) was added a solution of n-butyllithium in hexane (1.6 M, 8.51 mL) at −78° C., and the mixture was stirred at the same temperature for 30 min. (R)-Glycidyl butyrate (2.11 mL) was added to the mixture at −78° C. and the mixture was allowed to stand at room temperature for 4 hours. After quenching the reaction with the addition of aqueous ammonium chloride solution and dilution with ethyl acetate, the resulting mixture was washed with brine, dried over anhydrous sodium sulfate, filtered, and then concentrated in vacuo. Flash chromatography (silica, hexane:ethyl acetate=1:5) of the residue gave 5(R)-3-[4-[(1α,5α,6α)-6-(N-benzyl-N-t-butoxycarbonyl)amino-3-azabicyclo[3.1.0]hexan-3-yl]-3-fluorophenyl]-5-hydroxymethyloxazolidin-2-one (3.95 g).

¹H NMR (CDCl₃) δ 1.49 (s, 9H), 1.90 (s, 2H), 2.47 (s, 1H), 3.27 (d, J=8.8 Hz, 2H), 3.50-4.00 (m, 4H), 3.88 (dd, J=8.8, 6.8 Hz, 1H), 3.95 (t, J=8.8 Hz, 1H), 4.45 (s, 2H), 4.60-4.80 (m, 1H), 6.55 (t, J=9.3 Hz, 1H), 7.02 (dd, J=8.8, 2.4 Hz, 1H), 7.20-7.40 (m, 6H).

MS (EI⁺) m/z: 497 (M⁺).

### REFERENCE EXAMPLE 2

### 5(R)-Azidomethyl-3-[4-[(1α,5α,6α)-6-(N-t-butoxycarbonyl)amino-3-azabicyclo[3.1.0]hexan-3-yl]-3-fluorophenyl]oxazolidin-2-one

Step 1.

### 5(R)-3-[4-[(1α,5α,6α)-6-(N-t-butoxycarbonyl)amino-3-azabicyclo[3.1.0]hexan-3-yl]-3-fluorophenyl]-5-hydroxymethyloxazolidin-2-one

To a solution of 5(R)-3-[4-[(1α,5α,6α)-6-(N-benzyl-N-t-butoxycarbonyl)amino-3-azabicyclo[3.1.0]hexan-3-yl]-3-fluorophenyl]-5-hydroxymethyloxazolidin-2-one (4.41 g) in dichloromethane (40 mL) and methanol (15 mL) was added a solution of 4 N HCl in dioxane (21 mL), the mixture was stirred at room temperature for 9.5 hours, and then concentrated in vacuo. The residue was diluted with water, adjusted to pH 8 by the addition of saturated sodium hydrogencarbonate solution, and extracted with ethyl acetate. The organic extracts were washed with brine, dried over anhydrous magnesium sulfate, filtered, and then concentrated in vacuo to give 5(R)-3-[4-[(1α,5α,6α)-6-(N-benzyl)amino-3-azabicyclo[3.1.0]hexan-3-yl]-3-fluorophenyl]-5-hydroxymethyloxazolidin-2-one. This was used in the next step without further purification. A suspension of 5(R)-3-[4-[(1α,5α,6α)-6-(N-benzyl)amino-3-azabicyclo[3.1.0]hexan-3-yl]-3-fluorophenyl]-5-hydroxymethyloxazolidin-2-one and palladium catalyst (10% on charcoal, 400-mg) in dichloromethane (10 mL) and methanol (100 mL) was hydrogenated at 1 atm for 20 hours at room temperature. After filtration of the catalyst, the filtrate was concentrated in vacuo. To a solution of the residue in tetrahydrofuran (5 mL) was added triethylamine (2.0 mL) and di-t-butyl dicarbonate (1.90 g), the mixture was stirred at room temperature for 14 hours, and then concentrated in vacuo. Treatment with ethyl acetate and dichloromethane of the residue gave 5(R)-3-[4-[(1α,5α,6α)-6-(N-t-butoxycarbonyl)amino-3-azabicyclo[3.1.0]hexan-3-yl]-3-fluorophenyl]-5-hydroxymethyloxazolidin-2-one (3.05 g).

MS (EI⁺) m/z: 407 (M).

HRMS (EI⁺) for C₂₀H₂₆FN₃O₅ (M⁺): calcd, 407.1856; found, 407.1834.

Step 2.

### 5(R)-3-[4-[(1α,5α,6α)-6-(N-t-butoxycarbonyl)amino-3-azabicyclo[3.1.0]hexan-3-yl]-3-fluorophenyl]-5-(3-nitrobenzenesulfonyl)oxymethyloxazolidin-2-one

To a suspension of 5(R)-3-[4-[(1α,5α,6α)-6-(N-t-butoxycarbonyl)amino-3-azabicyclo[3.1.0]hexan-3-yl]-3-fluorophenyl]-5-hydroxymethyloxazolidin-2-one (204 mg) in tetrahydrofuran (5 mL) was added triethylamine (0.13 mL) and 3-nitrobenzenesulfonyl chloride (166 mg), the mixture was stirred at room temperature for 4 hours. The mixture was washed with brine, dried over anhydrous sodium sulfate, filtered, and then concentrated in vacuo to give 5(R)-3-[4-[(1α,5α,6α)-6-(N-t-butoxycarbonyl)amino-3-azabicyclo[3.1.0]hexan-3-yl]-3-fluorophenyl]-5-(3-nitrobenzenesulfonyl)oxymethyloxazolidin-2-one (293 mg).

MS (FAB⁺) m/z: 592 (MH⁺).

HRMS (FAB⁺) for C₂₆H₂₉FN₄O₉S (MH⁺): calcd, 592.1639; found, 592.1652.

Step 3.

### 5(R)-Azidomethyl-3-[4-[(1α,5α,6α)-6-CM-t-butoxycarbonyl)amino-3-azabicyclo[3.1.0]hexan-3-yl]-3-fluorophenyl]oxazolidin-2-one

The mixture of 5(R)-3-[4-[(1α,5α,6α)-6-(N-t-butoxycarbonyl)amino-3-azabicyclo[3.1.0]hexan-3-yl]-3-fluorophenyl]-5-(3-nitrobenzenesulfonyl)oxymethyloxazolidin-2-one (290 mg) and sodium azide (112 mg) in N,N-dimethylformamide (3 mL) was stirred at room temperature overnight. The mixture was diluted with dichloromethane and washed with water. The organic extracts were dried over anhydrous sodium sulfate, filtered, and then concentrated in vacuo to give 5(R)-azidomethyl-3-[4-[(1α,5α,6α)-6-(N-t-butoxycarbonyl)amino-3-azabicyclo[3.1.0]hexan-3-yl]-3-fluorophenyl]oxazolidin-2-one (197 mg).

MS (EI⁺) m/z: 432 (M⁺).

HRMS (EI⁺) for C₂₀H₂₅FN₆O₄ (M⁺): calcd, 432.1921; found, 432.1943.

### REFERENCE EXAMPLE 3

### 5(R)-3-[4-[(1α,5α,6α)-6-(N-Benzyl-N-t-butoxycarbonyl)amino 3 azabicyclo[3.1.0]hexan-3-yl]-3,5-difluorophenyl]-5-hydroxymethyloxazolidin-2-one

Step 1.

### 4-[(1α,5α,6α)-6-(N-t-Butoxycarbonyl)amino-3-azabicyclo[3.1.0]hexan-3-yl]-3,5-difluoronitrobenzene

The title compound 4-[(1α,5α,6α)-6-(N-t-butoxycarbonyl)amino-3-azabicyclo[3.1.0]hexan-3-yl]-3,5-difluoronitrobenzene (4.59 g) was prepared from (1α,5α,6α)-6-(N-t-butoxycarbonyl)amino-3-azabicyclo[3.1.0]hexane (3.50 g) and 3,4,5-trifluoronitrobenzene (3.20 g) in the same manner as described for REFERENCE EXAMPLE 1.

MS (EI⁺) m/z: 355 (M⁺).

HRMS (EI⁺) for C₁₆H₁₉F₂N₃O₄(M⁺): calcd, 355.1344; found, 355.1357.1

Step 2.

### 4-[(1α,5α,6α)-6-(N-Benzyl-N-t-butoxycarbonyl)amino-3-azabicyclo[3.1.0]hexan-3-yl]-3,5-difluoronitrobenzene

The title compound 4-[(1α,5α,6α)-6-(N-benzyl-N-t-butoxycarbonyl)amino-3-azabicyclo[3.1.0]hexan-3-yl]-3,5-difluoronitrobenzene (4.40 g) was prepared from 4-[(1α,5α,6α)-6-(N-t-butoxycarbonyl)amino-3-azabicyclo[3.1.0]hexan-3-yl]-3,5-difluoronitrobenzene (4.11 g) in the same manner as described for REFERENCE EXAMPLE 1.

¹H NMR (CDCl₃) δ 1.49 (s, 9H), 1.92 (s, 2H), 2.31 (s, 1H), 3.73-3.90 (m, 4H), 4.45 (s, 2H), 7.23-7.68 (m, 7H).

Step 3.

### 4-[(1α,5α,6α)-6-(N-Benzyl-N-t-butoxycarbonyl)amino-3-azabicyclo[3.1.0]hexan-3-yl]-1-benzyloxycarbonylamino-3,5-difluorobenzene

The title compound 4-[(1α,5α,6α)-6-(N-benzyl-N-t-butoxycarbonyl)amino-3-azabicyclo[3.1.0]hexan-3-yl]-1-benzyloxycarbonylamino-3,5-difluorobenzene (4.72 g) was prepared from 4-[(1α,5α,6α)-6-(N-benzyl-N-t-butoxycarbonyl)amino-3-azabicyclo[3.1.0]hexan-3-yl]-3,5-difluoronitrobenzene (4.40 g) in the same manner as described for REFERENCE EXAMPLE 1.

MS (FAB⁺) m/z: 550 (MH⁺).

HRMS (FAB⁺) for C₃₁H₃₄F₂N₃O₄ (MH⁺): calcd, 550.2517; found, 550.2507.

Step 4.

### 5(R)-3-[4-[(1α,5α,6α)-6-(N-Benzyl)amino-3-azabicyclo[3.1.0]hexan-3-yl]-3,5-difluorophenyl]-5-hydroxymethyloxazolidin-2-one

The title compound 5(R)-3-[4-[(1α,5α,6α)-6-N-benzyl)amino-3-azabicyclo[3.1.0]hexan-3-yl]-3,5-difluorophenyl]-5-hydroxymethyloxazolidin-2-one was prepared from 5(R)-3-[4-[(1α,5α,6α)-6-(N-benzyl-N-t-butoxycarbonyl)amino-3-azabicyclo[3.1.0]hexan-3-yl]-3,5-difluorophenyl]-5-hydroxymethyloxazolidin-2-one in the same manner as described for REFERENCE EXAMPLE 1.

MS (FAB⁺) m/z: 416 (MH⁺).

HRMS (FAB⁺) for C₂₂H₂₄F₂N₃O₃ (MH⁺): calcd, 416.1786; found, 416.1820.

### REFERENCE EXAMPLE 4

### 5(R)-3-[4-[(1α,5α,6α)-6-(N-t-Butoxycarbonyl)amino-3-azabicyclo[3.1.0]hexan-3-yl]-3,5-difluorophenyl]-5-hydroxymethyloxazolidin-2-one

The title compound 5(R)-3-[4-[(1α,5α,6α)-6-(N-t-butoxycarbonyl)amino-3-azabicyclo[3.1.0]hexan-3-yl]-3,5-difluorophenyl]-5-hydroxymethyloxazolidin-2-one (2.44 g) was prepared from 5(R)-3-[4-[(1α,5α,6α)-6-(N-benzyl)amino-3-azabicyclo[3.1.0]hexan-3-yl]-3,5-difluorophenyl]-5-hydroxymethyloxazolidin-2-one (3.59 g) in the same manner as described for REFERENCE EXAMPLE 1.

MS (FAB⁺) m/z: 426 (MH⁺).

HRMS (FAB⁺) for C₂₀H₂₆F₂N₃O₅ (MH⁺): calcd, 426.1841; found, 426.1805.

### REFERENCE EXAMPLE 5

### 5(R)-Azidomethyl-3-[4-[(1α,5α,6α)-6-(N-t-butoxycarbonyl)amino-3-azabicyclo[3.1.0]hexan-3-yl]-3,5-difluorophenyl]oxazolidin-2-one

To a solution of 5(R)-3-[4-[(1α,5α,6α)-6-(N-t-butoxycarbonyl)amino-3-azabicyclo[3.1.0]hexan-3-yl]-3,5-difluorophenyl]-5-hydroxymethyloxazolidin-2-one (749 mg) in tetrahydrofuran (30 mL) were successively added triethylamine (0.32 mL) and methanesulfonyl chloride (0.18 mL) at 0° C., and the mixture was stirred at the same temperature for 2 hours. The mixture was diluted with ethyl acetate, and washed with water and brine. The organic extracts were dried over anhydrous magnesium sulfate, filtered, and then concentrated in vacuo to give 5(R)-3-[4-[(1α,5α,6α)-6-(N-t-butoxycarbonyl)amino-3-azabicyclo[3.1.0]hexan-3-yl]-3-fluorophenyl]-5-methanesulfonyloxymethyloxazolidin-2-one. This was used in the next step without further purification. The mixture of crude 5(R)-3-[4-[(1α,5α,6α)-6-N-t-butoxycarbonyl)amino-3-azabicyclo[3.1.0]hexan-3-yl]-3-fluorophenyl]-5-methanesulfonyloxymethyloxazolidin-2-one thus obtained and sodium azide (172 mg) in N,N-dimethylformamide (30 mL) was heated at 70° C. for 5.5 hours, and then concentrated in vacuo. The residue was diluted with ethyl acetate and washed with water and brine. The organic extracts were dried over anhydrous magnesium sulfate, filtered, and then concentrated in vacuo to give 5(R)-azidomethyl-3-[4-[(1α,5α,6α)-6-(N-t-butoxycarbonyl)amino-3-azabicyclo[3.1.0]hexan-3-yl]-3-fluorophenyl]oxazolidin-2-one (623 mg).

MS (EI⁺) m/z: 450 (M⁺).

HRMS (EI⁺) for C₂₀H₂₄F₂N₆O₄ (M⁺): calcd, 450.1827; found, 450.1850.

### REFERENCE EXAMPLE 6

### (1α,5α,6α)-6-[(t-Butyldiphenylsilyl)oxy]methylbicyclo[3.1.0]hex-2-ene

To a solution of (1α,5α,6α)-bicyclo[3.1.0]hex-2-en-6-methanol (11.0 mg) in dichloromethane (0.4 mL) was added t-butyldiphenylsilyl chloride (32 μL), triethylamine (35 μL), and 4-(dimethylamino)pyridine (24.4 mg), the mixture was stirred at room temperature for 3 hours. After quenching the reaction by the addition of 1 N hydrochloric acid, the mixture was extracted with ethyl acetate. The organic extracts were washed with water, sodium hydrogencarbonate solution, and brine, dried over anhydrous sodium sulfate, and then concentrated in vacuo. Flash chromatography (silica, hexane:ethyl acetate=50:1) of the residue gave (1α,5α,6α)-6-[(t-butyldiphenylsilyl)oxy]methylbicyclo[3.1.0]hex-2-ene (28.3 mg).

¹H NMR (CDCl₃) δ 0.47-0.52 (m, 1H), 1.05 (s, 9H), 1.40-1.43 (m, 1H), 1.67-1.69 (m, 1H), 2.27-2.32 (m, 1H), 2.50-2.60 (m, 1H), 3.50-3.60 (m, 2H), 5.37-5.39 (m, 1H), 5.80-5.90 (m, 1H), 7.36-7.44 (m, 6H), 7.67-7.69 (m, 4H).

MS (EI⁺) m/z: 348 (M⁺).

### REFERENCE EXAMPLE 7

### (1α,5α,6α)-6-[(t-Butyldiphenylsilyl)oxy]methyl-3-hydroxybicyclo[3.1.0]hexane Isomer A and B

To a solution of (1α,5α,6α)-6-[(t-butyldiphenylsilyl)oxy]methylbicyclo[3.1.0]hex-2-ene (2.79 g) in tetrahydrofuran (28 mL) was added borane-methyl sulfide complex (927 μL) at 0° C., the mixture was stirred at room temperature for 1.5 hours. The resulting solution was added water (22 mL), 2.5 N sodium hydroxide solution (4.8 mL), and hydrogen peroxide solution (30%, 1.36 mL) at 0° C., the mixture was stirred at room temperature for 1 hour. After dilution the mixture with water, the resulting mixture was extracted with ethyl acetate. The organic extracts were washed with brine, dried over anhydrous sodium sulfate, and then concentrated in vacuo. Flash chromatography (silica, hexane:ethyl acetate=2:1) of the residue gave the two isomers of (1α,5α,6α)-6-[(t-butyldiphenylsilyl)oxy]methyl-3-hydroxybicyclo[3.1.0]hexane (2.38 g).

Isomer A

¹H NMR (CDCl₃) δ 0.70-0.75 (m, 1H), 1.03 (s, 9H), 1.00-1.70 (m, 4H), 2.11 (dd, J=12.7, 6.8 Hz, 2H), 3.43 (d, J=6.4 Hz, 2H), 3.90-4.00 (m, 1H), 7.36-7.44 (m, 6H), 7.65-7.70 (m, 4H).

MS (CI⁺) m/z: 367 (MH⁺).

Isomer B

¹H NMR (CDCl₃) δ 1.04 (s, 9H), 1.00-1.10 (m, 2H), 1.26-1.31 (m, 1H), 1.68 (d, J=14.2 Hz, 2H), 2.00-2.10 (m, 2H), 3.51 (d, J=6.4 Hz, 2H), 4.35 (t, J=6.4 Hz, 1H), 7.35-7.44 (m, 6H), 7.66-7.70 (m, 4H).

MS (CI⁺) m/z: 367 (MH⁺).

### REFERENCE EXAMPLE 8

### (1α,5α,6α)-6-[(t-Butyldiphenylsilyl)oxy]methyl-3-oxobicyclo[3.1.0]hexane

To a solution of (1α,5α,6α)-6-[(t-butyldiphenylsilyl)oxy]methyl-3-hydroxybicyclo[3.1.0]hexane (2.38 g) in dimethyl sulfoxide (24 mL) was added 1-hydroxy-1, 2-benziodoxol-3(1H)-one 1-oxide (2.73 g), the mixture was stirred at room temperature for 5.5 hours. After addition of ethyl acetate and water, insoluble materials were filtered off. The organic extracts were washed with water and brine, dried over anhydrous sodium sulfate, and then concentrated in vacuo. Flash chromatography (silica, hexane:ethyl acetate=5:1) of the mixture gave (1α,5α,6α)-6-[(t-butyldiphenylsilyl)oxy]methyl-3-oxobicyclo[3.1.0]hexane (1.82 g).

¹H NMR (CDCl₃) δ 0.60-0.65 (m, 1H), 1.04 (s, 9H), 1.38-1.40 (m, 2H), 2.14 (dd, J=18.6, 2.0 Hz, 2H), 2.50-2.60 (m, 2H), 3.62 (d, J=5.9 Hz, 2H), 7.40-7.50 (m, 6H), 7.65-7.68 (m, 4H).

MS (EI⁺) m/z: 364 (M⁺).

### REFERENCE EXAMPLE 9

### (1α,5α,6α)-6-[(t-butyldiphenylsilyl)oxy]methyl-3-[(trifluoromethanesulfonyl)oxy]bicyclo[3.1.0]hex-2-ene

To a solution of (1α,5α,6α)-6-[(t-butyldiphenylsilyl)oxy]methyl-3-oxobicyclo[3.1.0]hexane (365 mg) in tetrahydrofuran (2 mL) was added a solution of lithium diisopropylamide (2M, 650 μL) at −78° C., the mixture was stirred at the same temperature for 30 minutes. The resulting mixture was added a solution of N-phenylbis(trifluoromethanesulfonimide) (393 mg) in tetrahydrofuran (2 mL) at −78° C., the mixture was stirred at room temperature for 17 hours, and then concentrated in vacuo. Flash chromatography (silica, hexane:ethyl acetate=25:1) of the residue gave (1α,5α,6α)-6-[(t-butyldiphenylsilyl)oxy]methyl-3-[(trifluoromethanesulfonyl)oxy]bicyclo[3.1.0]hex-2-ene (313 mg).

¹H NMR (CDCl₃) δ 0.77-0.82 (m, 1H), 1.04 (s, 9H), 1.30-1.50 (m, 1H), 1.60-1.70 (m, 1H), 2.48-2.53 (m, 1H), 2.79-2.85 (m, 1H), 3.50-3.60 (m, 2H), 5.78-5.79 (m, 1H), 7.40-7.50 (m, 6H), 7.60-7.70 (m, 4H).

### REFERENCE EXAMPLE 10

### (1α,5α,6α)-6-[(t-Butyldiphenylsilyl)oxy]methyl-3-(4,4,5,5-tetramethyl-1,3,2-dioxaborolyl)bicyclo[3.1.0]hex-2-ene

The mixture of (1α,5α,6α)-6-[(t-butyldiphenylsilyl)oxy]methyl-3-[(trifluoromethanesulfonyl)oxy]bicyclo[3.1.0]hex-2-ene (100 mg), bis(pinacolato)diboron (56.3 mg), potassium phenoxide (39.9 mg), bis(triphenylphosphine)dichloropalladium (7.1 mg) and triphenylphosphine (5.3 mg) in toluene (2 mL) was stirred at 50° C. for 2.5 hours. Flash chromatography (silica, hexane:ethyl acetate=20:1) of the mixture gave (1α,5α,6α)-6-[(t-butyldiphenylsilyl)oxy]methyl-3-(4,4,5,5-tetramethyl-1,3,2-dioxaborolyl)bicyclo[3.1.0]hex-2-ene (63.0 mg).

MS (EI⁺) m/z: 474 (M⁺).

HRMS (EI⁺) for C₂₉H₃₉BO₃Si (M⁺): calcd, 474.2762; found, 474.2737.

### REFERENCE EXAMPLE 10

### 5(R)-Azidomethyl-3-(4-iodophenyl)oxazolidin-2-one

The title compound 5(R)-azidomethyl-3-(4-iodophenyl)oxazolidin-2-one (95.4 g) was prepared from 5(R)-3-(4-iodophenyl)-5-hydroxymethyloxazolidin-2-one (70.0 g) in the same manner as described for REFERENCE EXAMPLE 5.

MS (EI⁺) m/z: 344 (M⁺).

HRMS (Et for C₁₀H₉IN₄O₂ (M⁺): calcd, 343.9770; found, 343.9740.

### REFERENCE EXAMPLE 11

### 1-[5(R)-3-(4-Iodophenyl)-2-oxooxazolidin-5-ylmethyl]-1,2,3-triazole

The title compound 1-[5(R)-3-(4-iodophenyl)-2-oxooxazolidin-5-ylmethyl]-1,2,3-triazole (62.5 mg) was prepared from 5(R)-azidomethyl-3-(4-iodophenyl)oxazolidin-2-one (100 mg) in the same manner as described for EXAMPLE 1.

MS (EI⁺) m/z: 370 (M⁺).

HRMS (EI⁺) for C₁₂H₁₁N₄O₂ (M⁺): calcd, 369.9927; found, 369.9919.

### REFERENCE EXAMPLE 12

### 5(R)-Azidomethyl-3-(3-fluoro-4-iodophenyl)oxazolidin-2-one

The title compound 5(R)-azidomethyl-3-(3-fluoro-4-iodophenyl)oxazolidin-2-one (2.18 g) was prepared from 5(R)-3-(3-fluoro-4-iodophenyl)-5-hydroxymethyloxazolidin-2-one (2.00 g) in the same manner as described for REFERENCE EXAMPLE 5.

MS (EI⁺) m/z: 344 (M⁺).

HRMS (EI⁺) for C₁₀H₉IN₄O₂ (M⁺): calcd, 343.9770; found, 343.9740.

### REFERENCE EXAMPLE 13

### 1-[5(R)-3-(3-Fluoro-4-iodophenyl)-2-oxooxazolidin-5-ylmethyl]-1,2,3-triazole

The title compound 1-[5(R)-3-(3-fluoro-4-iodophenyl)-2-oxooxazolidin-5-ylmethyl]-1,2,3-triazole (1.70 g) was prepared from 5(R)-azidomethyl-3-(3-fluoro-4-iodophenyl)oxazolidin-2-one (2.18 g) in the same manner as described for EXAMPLE 1.

MS (EI⁺) m/z: 388 (M⁺).

HRMS (EI⁺) for C₁₂H₁₀FIN₄O₂ (M⁺): calcd, 387.9833; found, 387.9835.

Antibacterial Activity

The pharmaceutically-acceptable compounds of the present invention are useful antibacterial agents having a good spectrum of activity in vitro against standard bacterial strains, which are used to screen for activity against pathogenic bacteria. Notably, the pharmaceutically-acceptable compounds of the present invention show activity against vancomycin-resistant enterococci, streptococci including penicillin-resistant S. pneumoniae, methicillin-resistant S. aureus, M. catarrhalis, and C. pneumoniae. The antibacterial spectrum and potency of a particular compound may be determined in a standard test system.

The following in vitro results were obtained based on an agar dilution method except for C. pneumoniae. The activity is presented as the minimum inhibitory concentration (MIC).

S. aureus and M. catarrhalis were tested on Mueller-Hinton agar, using an approximate inoculum of 1×10⁴ cfu/spot an incubation temperature of 35° C. for 24 hours. The MIC was defined as the lowest concentration at which no visible bacterial growth was observed.

Streptococci and enterococci were tested on Mueller-Hinton agar supplemented with 5% defibrinated horse blood, using an approximate inoculum of 1×10⁴ cfu/spot an incubation temperature of 35° C. in an atmosphere of 5% CO₂ for 24 hours. The MIC was defined as the lowest concentration at which no visible bacterial growth was observed.

C. pneumoniae was tested using minimum essential medium supplemented with 10% heat-inactivated fetal bovine serum, 2 mM L-glutamine, 1 mg/ml cycloheximide and non essential amino acid. HeLa 229 cells were inoculated with 10⁴ inclusion-forming units of C. pneumoniae strain per mL. Infected cells were incubated with test compounds in complete medium at 35° C. in an atmosphere of 5% CO₂ for 72 hours. Cells monolayers were fixed in methanol, stained for chlamydial inclusions with an fluorescein-conjugated anti-Chlamydia monoclonal antibody, and were observed with fluorescence microscope. The MIC was defined as the lowest concentration at which no inclusion was observed.

| | MIC (μg/ml) | | |
|---|---|---|---|
| Strains | example 2 | example 8 | Linezolid |
| Staphylococcus aureus | | | |
| Smith | 0.125 | 0.125 | 1 |
| CR | 2 | 1 | 16 |
| MR | 0.25 | 0.06 | 1 |
| Streptococcus pneumoniae | | | |
| IID553 | 0.25 | 0.5 | 2 |
| PRQR | 0.25 | 0.25 | 1 |
| Streptococcus pyogenes | | | |
| IID692 | 0.25 | 0.125 | 1 |
| Enterococcus faecium | | | |
| VRQR | 1 | 0.25 | 2 |
| Moraxella catarrhalis | | | |
| ATCC25238 | 1 | 1 | 4 |

CR = chloramphenicol resistant
MR = methicillin resistant
PRQR = penicillin resistant, quinolone resistant
VRQR = vancomycin resistant, quinolone resistant
NT = not tested

The invention described herein is exemplified by the following non-limiting examples. The compound data is designated in accordance to General Guidelines for Manuscript Preparation, J. Org. Chem. Vol. 66, pg. 19A, Issue 1, 2001.

1. The present invention relates to compounds of formula I: 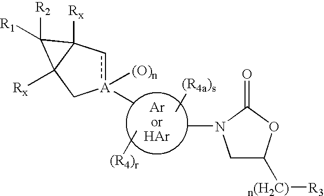
its enantiomer, diastereomer, or pharmaceutically acceptable salt, hydrate or prodrug thereof wherein:
R₁ and R₂ independently represent
hydrogen, NR₅R₆, CR₇R₈R₉, C(R)₂OR₁₄, CH₂NHR₁₄, C(═O)R₁₃, C(═NOH)H, C(═NOR₁₃)H, C(═NOR₁₃)R₁₃, C(═NOH)R₁₃, C(═O)N(R₁₃)₂, C(═NOH)N(R₁₃)₂, NHC(═X₁)N(R₁₃)₂, (C═NH)R₇, N(R₁₃)C(═X₁)N(R₁₃)₂, COOR₁₃, SO₂R₁₄, N(R₁₃)SO₂R₁₄, N(R₁₃)COR₁₄, (C₁₋₆alkyl)CN, CN, CH═C(R)₂, C(R₄)₂X₁SiR₁₆, (CH₂)ₚOH, C(═O)CHR₁₃, C(═NR₁₃)R₁₃, NR₁₀C(═X₁)R₁₃; or C₅₋₁₀ heterocycle optionally substituted with 1-3 groups of R₇, which may be attached through either a carbon or a heteroatom;
A represents C (when [symbol] is present), CH or N (when [symbol] is not present); [symbol]
represents a bond; 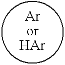
represents aryl or heteroaryl, provided that in the case of a heteroaryl, a cyclopropyl is not attached to a nitrogen atom on the ring;
Rₓ represents hydrogen or C₁₋₆ alkyl;
R₃ represents 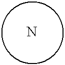
which is an optionally substituted aromatic heterocyclic group containing at least one nitrogen in the ring and which is attached through a bond on any N, and which is unsubstituted or contains 1 to 3 substituents of R₇
R₄ and R₄ₐ independently represent
hydrogen,
halogen,
C₁₋₆ alkoxy, or
C₁₋₆ alkyl
r and s independently are 1-3, with the provision that when (R₄ₐ)ₛ and (R₄)ᵣ are attached to an Ar or HAr ring the sum of r and s is less than or equal to 4;
R₅ and R₆ independently represent
hydrogen, C₁₋₆ alkyl optionally substituted with 1-3 groups of halogen, CN, OH, C₁₋₆ alkoxy, amino, imino, hydroxyamino, alkoxyamino, C₁₋₆ acyloxy, C₁₋₆ alkylsulfenyl, C₁₋₆ alkylsulfinyl, C₁₋₆ alkylsulfonyl, aminosulfonyl, C₁₋₆ alkylaminosulfonyl, C₁₋₆ dialkylaminosulfonyl, 4-morpholinylsulfonyl, phenyl, pyridine, 5-isoxazolyl, ethylenyloxy, or ethynyl, said phenyl and pyridine optionally substituted with 1-3 halogen, CN, OH, CF₃, C₁₋₆ alkyl or C₁₋₆ alkoxy; C₁₋₆ acyl optionally substituted with 1-3 groups of halogen, OH, SH, C₁₋₆ alkoxy, naphthalenoxy, phenoxy, amino, C₁₋₆ acylamino, hydroxylamino, alkoxylamino, C₁₋₆ acyloxy, aralkyloxy, phenyl, pyridine, C₁₋₆ alkylcarbonyl, C₁₋₆ alkylamino, C₁₋₆ dialkylamino, C₁₋₆ hydroxyacyloxy, C₁₋₆ alkylsulfenyl, phthalimido, maleimido, succinimido, said phenoxy, phenyl and pyridine optionally substituted with 1-3 groups of halo, OH, CN, C₁₋₆ alkoxy, amino, C₁₋₆ acylamino, CF₃ or C₁₋₆ alkyl;
C₁₋₆ alkylsulfonyl optionally substituted with 1-3 groups of halogen, OH, C₁₋₆ alkoxy, amino, hydroxylamino, alkoxylamino, C₁₋₆ acyloxy, or phenyl; said phenyl optionally substituted with 1-3 groups of halo, OH, C₁₋₆ alkoxy, amino, C₁₋₆ acylamino, CF₃ or C₁₋₆ alkyl; arylsulfonyl optionally substituted with 1-3 of halogen, C₁₋₆ alkoxy, OH or C₁₋₆ alkyl;
C₁₋₆ alkoxycarbonyl optionally substituted with 1-3 of halogen, OH, C₁₋₆ alkoxy, C₁₋₆ acyloxy, or phenyl, said phenyl optionally substituted with 1-3 groups of halo, OH, C₁₋₆ alkoxy, amino, C₁₋₆ acylamino, CF₃ or C₁₋₆ alkyl; aminocarbonyl, C₁₋₆ alkylaminocarbonyl or C₁₋₆ dialkylaminocarbonyl, said alkyl groups optionally substituted with 1-3 groups of halogen, OH, C₁₋₆ alkoxy or phenyl, five to six membered heterocycles optionally substituted with 1-3 groups of halogen, OH, CN, amino, C₁₋₆ acylamino, C₁₋₆ alkylsulfonylamino, C₁₋₆ alkoxycarbonylamino, C₁₋₆ alkoxy, C₁₋₆ acyloxy or C₁₋₆ alkyl, said alkyl optionally substituted with 1-3 groups of halogen, or C₁₋₆ alkoxy;
C₃₋₆ cycloalkylcarbonyl optionally substituted with 1-3 groups of halogen, OH, C₁₋₆ alkoxy or CN; benzoyl optionally substituted with 1-3 groups of halogen, OH, C₁₋₆ alkoxy, C₁₋₆ alkyl, CF₃, C₁₋₆ alkanoyl, amino or C₁₋₆ acylamino; pyrrolylcarbonyl optionally substituted with 1-3 of C₁₋₆ alkyl; C₁₋₂ acyloxyacetyl where the acyl is optionally substituted with amino, C₁₋₆ alkylamino, C₁₋₆ dialkylamino, 4-morpholino, 4-aminophenyl, 4-(dialkylamino)phenyl, 4-(glycylamino)phenyl; or R₅ and R₆ taken together with any intervening atoms can form a 3 to 7 membered heterocyclic ring containing carbon atoms and 1-2 heteroatoms independently chosen from O, S, SO, SO₂, N, or NR₈;
R₇ represent
hydrogen, halogen, CN, CO₂R, CON(R)₂, CHO, CH₂NHAc, C(═NOR), OH, C₁₋₆ alkoxy, C₁₋₆ alkyl, alkenyl, (CH₂)ₙamino, (CH₂)ₙC1-6 alkylamino, C₁₋₆ dialkylamino, hydroxylamino or C₁₋₂ alkoxyamino all of which can be optionally substituted on the nitrogen with C₁₋₆ acyl, C₁₋₆ alkylsulfonyl or C₁₋₆ alkoxycarbonyl, said acyl and alkylsulfonyl optionally substituted with 1-2 of halogen or OH;
R₈ and R₉ independently represents
H, CN,
C₁₋₆ alkyl optionally substituted with 1-3 halogen, CN, OH, C₁₋₆ alkoxy, C₁₋₆ acyloxy, or amino,
phenyl optionally substituted with 1-3 groups of halogen, OH, C₁₋₆ alkoxy; or
R₇ and R₈ taken together can form a 3-7 membered carbon ring optionally interrupted with 1-2 heteroatoms chosen from O, S, SO, SO₂, NH, and NR₈;
X₁ represents O, S or NR₁₃, NCN, NCO₂R₁₆, or NSO₂R₁₄
R₁₀ represents hydrogen, C₁₋₆ alkyl or CO₂R₁₅;
Each R₁₃ represents independently hydrogen, C₁₋₆ alkyl, C₆₋₁₀ aryl, NR₅R₆, SR₈, S(O)R₈, S(O)₂R₈, CN, OH, C₁₋₆ alkylS(O)R, C₁₋₆ alkoxycarbonyl, hydroxycarbonyl, C₁₋₆ acyl, C₃₋₇ membered carbon ring optionally interrupted with 1-4 heteroatoms chosen from O, S, SO, SO₂, NH and NR₈ where said C₁₋₆ alkyl, aryl or C₁₋₆ acyl groups may be independently substituted with 0-3 halogens, hydroxy, N(R)₂, CO₂R, C₆₋₁₀ aryl, C₅₋₁₀ heteroaryl, or C₁₋₆ alkoxy groups;
When two R₁₃ groups are attached to the same atom or two adjacent atoms they may be taken together to form a 3-7 membered carbon ring optionally interrupted with 1-2 heteroatoms chosen from O, S, SO, SO₂, NH, and NR₈;
R represents hydrogen or C₁₋₆ alkyl;
R₁₄ represents amino, C₁₋₆ alkyl, C₁₋₆ haloalkyl, five to six membered heterocycles or phenyl, said phenyl and heterocycles optionally substituted with 1-3 group of halo, C₁₋₆ alkoxy, C₁₋₆ acylamino, or C₁₋₆ alkyl, hydroxy and/or amino, said amino and hydroxy optionally protected with an amino or hydroxy protecting group;
R₁₅ is C₁₋₆ alkyl or benzyl said benzyl optionally substituted with 1-3 groups of halo, OH, C₁₋₆ alkoxy, amino, C₁₋₆ acylamino, or C₁₋₆ alkyl;
R₁₆ is hydrogen, C₅₋₁₀heteroaryl, C₆₋₁₀aryl, said heteroaryl and aryl optionally substituted with 1-3 groups of R₇;
m, n, p and q represents 0-1.

2. A compound according to claim 1 wherein R₁ and R₂ independently represent H, NR₅R₆, CN, OH, C(R)₂OR₁₄, NHC(═X1)N(R₁₃)₂, C(═NOH)N(R₁₃)₂, NR₁₀C(═X₁)R₁₃ or CR₇R₈R₉.

3. A compound according to claim 2 wherein 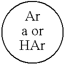
is phenyl, pyridine, pyrimidine, or piperidine.

4. A compound according to claim 3 wherein one of R₁ and R₂ is H and the other is NR₅R₆; H and the other is CN; or H and the other is NR₁₀C(═X₁)R₁₃.

5. A compound according to claim 4 wherein A is C, [symbol] is present, and Z=(O)ₙ where n=0; A is C, [symbol] is not present and Z=H, OH or halogen or A is N, [symbol] is not present and Z=(O)ₙ where n=1.

6. A compound according to claim 5 wherein R₃ is 1,2,3-triazole, 1,2,4-triazole, 1,2,5-triazole, tetrazole, pyrazole, or imidazole, any of which may contain 1 to 3 substitutents of R₇.

7. A compound which is:
1-[5(R)-3-[4-[(1α,5α,6α)-6-amino-3-azabicyclo[3.1.0]hexan-3-yl]-3-fluorophenyl]-2-oxooxazolidin-5-ylmethyl]-1,2,3-triazole,
1-[5(R)-3-[4-[(1α,5α,6α)-6-amino-3-azabicyclo[3.1.0]hexan-3-yl]-3,5-difluorophenyl]-2-oxooxazolidin-5-ylmethyl]-1,2,3-triazole,
1-[5(R)-3-[4-[(1α,5α,6α)-6-[(t-butyldiphenylsilyl)oxy]methylbicyclo[3.1.0]hex-2-en-3-yl]phenyl]-2-oxooxazolidin-5-ylmethyl]-1,2,3-triazole,
1-[5(R)-3-[4-[(1α,5α,6α)-6-[(t-butyldiphenylsilyl)oxy]methylbicyclo[3.1.0]hex-2-en-3-yl]-3-fluorophenyl]-2-oxooxazolidin-5-ylmethyl]-1,2,3-triazole,
1-[5(R)-3-[4-[(1α,5α,6α)-6-hydroxyoxymethylbicyclo[3.1.0]hex-2-en-3-yl]phenyl]-2-oxooxazolidin-5-ylmethyl]-1,2,3-triazole,
1-[5(R)-3-[3-fluoro-4-[(1α,5α,6α)-6-hydroxyoxymethylbicyclo[3.1.0]hex-2-en-3-yl]phenyl]-2-oxooxazolidin-5-ylmethyl]-1,2,3-triazole,
1-[5(R)-3-[4-[(1α,5α,6α)-6-cyanobicyclo[3.1.0]hex-2-en-3-yl]phenyl]-2-oxooxazolidin-5-ylmethyl]-1,2,3-triazole,
1-[5(R)-3-[4-[(1α,5α,6α)-6-cyanobicyclo[3.1.0]hex-2-en-3-yl]-3-fluorophenyl]-2-oxooxazolidin-5-ylmethyl]-1,2,3-triazole,
or its enantiomer, diastereomer, or pharmaceutically acceptable salt, hydrate or prodrug thereof.

8. A pharmaceutical composition comprised of a compound in accordance with claim 1 in combination with a pharmaceutically acceptable carrier and optionally a in combination with a vitamin selected from the group consisting vitamin B2, vitamin B6, vitamin B12 and folic acid.

9. A method of treating or preventing a bacterial infection in a mammalian patient in need thereof, comprising administering to said patient an effective amount of a compound of claim 1.

10. A method of treating or preventing bacterial infection or an oxazolidinone-associated side effect by administering an effective amount of a compound of formula I of claim 1 and an effective amount of one or more of a vitamin selected from the group consisting of vitamin B2, vitamin B6, vitamin B12 and folic acid to a patient in need thereof.

11. A method according to claim 10 for treating or preventing oxazolidinone-associated normocytic anemia, peripheral sensory neuropathy, sideroblastic anemia, peripheral sensory neuropathy, optic neuropathy, seizures, thrombocytopenia, cheilosis, hypo-regenerative anemia, megaloblastic anemia and seborrheic dermatitis by administering an effective amount of vitamin B2 to a patient in need thereof.
